(19) United States
(12) Patent Application Publication
(10) Pub. No.: US 2017/0189017 A1
(43) Pub. Date: Jul. 6, 2017

(54) CURLED SUTURE DEVICE APPARATUS AND METHOD

(71) Applicant: Thomas Salvant, Mandeville, LA (US)

(72) Inventor: Thomas Salvant, Mandeville, LA (US)

(21) Appl. No.: 15/394,578

(22) Filed: Dec. 29, 2016

Related U.S. Application Data
(60) Provisional application No. 62/274,145, filed on Dec. 31, 2015.

(51) Int. Cl. A61B 17/06; A61B 1/313; A61B 17/04

(52) U.S. Cl. CPC A61B 17/06166; A61B 17/0469; A61B 17/0467; A61B 17/06066; A61B 1/3132; A61B 2017/0477; A61B 2017/0608; A61B 2017/06171; A61B 2017/00004

(57) ABSTRACT

The present invention provides a curled suture device to minimize interference and obstructions during intricate surgeries, such as laparoscopic surgeries. The curled suture device has a needle and a significantly shorter suture and thread than the sutures currently available for use. Towards the end of the suture, opposite the side of the needle, a stopping mechanism is featured in conjunction with an angled, less flexible, portion of thread. The stopping mechanism acts to halt the movement of the thread as the needle and thread is pulled through the suture point, securing the remainder of the suture thread on the first side of the tissue being sutured. On this side of the stopping mechanism, a curled portion of thread is featured, such that the suture thread curls into a corkscrew configuration that loops around several times before coming to an end. This corkscrew configuration allows for a ready-made knot in the suture device, to aid in minimizing interference and obstruction during surgery. The length of the entire device, from one tip of the needle to the end of the curled suture, is of a length significantly shorter than typical needles and suture, which may stretch up to several feet long, which also aids in reducing interference and obstruction during the surgical process.

### BACKGROUND OF THE INVENTION

present invention provides a curled suture device to minimize interference and obstructions during intricate surgeries, such as laparoscopic surgeries. The curled suture device has a needle and a significantly shorter suture and thread than the sutures currently available for use. Towards the end of the suture, opposite the side of the needle, a stopping mechanism is featured in conjunction with an angled, less flexible, portion of thread. The stopping mechanism acts to halt the movement of the thread as the needle and thread is pulled through the suture point, securing the remainder of the suture thread on the first side of the tissue being sutured. On this side of the stopping mechanism, a curled portion of thread is featured, such that the suture thread curls into a corkscrew configuration that loops around several times before coming to an end. This corkscrew configuration allows for a ready-made knot in the suture device, to aid in minimizing interference and obstruction during surgery. The length of the entire device, from one tip of the needle to the end of the curled suture, is of a length significantly shorter than typical needles and suture, which may stretch up to several feet long, which also aids in reducing interference and obstruction during the surgical process.

In laparoscopic surgery or other similar procedures, the surgeon may be required to work on the inside of a particular cavity of the body of the patient. In working in this cavity, there may be instruments inserted into the patient to allow the surgeon to perform the procedure, such as a camera, instruments holding the cavity open in an appropriate manner, and multiple sutures and associated thread to allow for repair of the interior tissue. Use of these instruments ultimately crowds the surgeon's working space, and interferes with a smooth and efficient surgical procedure.

By providing a curled suture device with a ready-made knot, the surgeon is able to perform the suturing with only one hand, while his or her second hand may secure, for example, the camera utilized in the procedure. Further, by eliminating the need to create a knot from standard thread, the thread may be significantly shortened from needle to tip, which frees space within the surgical cavity. The curled portion also centers the suturing area in the appropriate section of the surgical cavity, which allows the surgeon to operate within the scope of his or her view within the surgical cavity, and also allows for fewer adjustments to the position of the camera, which in turn decreases disruption of both the procedure and physicality of the patient. The stopping mechanism featured in the curled suture device of the present invention further facilitates these advantages, as it allows for the surgeon to more easily create the knot by ensuring the curled section of the suture remains in the appropriate place during the suturing process. Additionally, in allowing for the surgeon to control both the suture and sutured tissue with a single hand, the surgeon is able to adjust the final suture spot as necessary without additional disruption of the tissue and surgical cavity.

As outlined below, various references provide for curls within a suture device. However, the present art does not provide for a stopper on the suture to more effectively create the knot in the desired area of suturing, and therefore does not aid the surgeon in the laparoscopic procedure. The prior art additionally does not contemplate the varied stopper mechanisms that should be taken into account when tailoring the suture device for specific surgical procedures, as outlined in more detail below.

U.S. Publ. No. 2009/0216268, published Aug. 27, 2009 by Gideon G. Panter, discloses a suture for facilitating tying of knots that includes a length of suture material fixed to a needle. At least part of the length of the suture material is resiliently biased to a coiled state which is straightenable under applied tension, but which returns to its coiled state when released. In use, part of the suture material is drawn through a region of tissue so that portions of the suture material lie on opposite ends of the tissue. A knot is then formed in the suture material by passing one of the portions through at least one resiliently-formed coil formed in the other of the portions, and pulling tight the suture material.

The Panter suture disclosed above discusses the use of a coiled thread to assist in the creation of a knot, but does not provide for a stopper mechanism built into the suture device. Such a stopper would be necessary to create the knot in the appropriate spot of the suture device, and would also assist the surgeon in pulling the tissue regions together when creating the knot in the suture material.

U.S. Pat. No. 5,454,834, issued Oct. 3, 1995 to Manfred Boebel et al., discloses a surgical suture material provided with a thread and, in some cases, a needle to allow formation of a knot with the greatest possible security while expending little effort, even in situations where space is limited—for example, during an endoscopic operation. The suture material has at least one inherently stable coil, loop, or similar preformed featured in at least one initial preformed section of its length. Another section of the thread or an end of the thread can be threaded or guided through this preformed feature for the purpose of forming a loop and/or knot. The preformed section can take the form of a preformed feature with an approximately spiral shape, for example.

However, the Boebel patent, like the Panter suture disclosed above, does not provide for a stopping mechanism to assist in the knot creation and suturing activity of the surgeon during the suturing process. Further, the Boebel patent requires multiple coils in the thread, which simply serves to further crowd the suturing space instead of simplifying and streamlining the suturing process, as is intended by the present invention.

There is accordingly a need for a curled suture device that may be used in laparoscopic procedures that aids the surgeon in reducing interference during the surgical procedure by provision of a ready-made knot within the suture, which is in part facilitated by a stopping mechanism in the suture that aids in the creation of the knot, and also secures the pre-knot curled section in the appropriate area during the procedure. In allowing for the surgeon to suture the appropriate tissue with only one hand, the surgeon may also easily adjust the location of the suture and upcoming knot within the surgical cavity, which allows for reduced interference within the surgical cavity.

### SUMMARY OF THE INVENTION

The present invention provides a curled suture device to minimize interference and obstructions during intricate surgeries, such as laparoscopic surgeries. The curled suture device has a needle and a significantly shorter suture and thread than the sutures currently available for use. Towards the end of the suture, opposite the side of the needle, a stopping mechanism is featured in conjunction with an angled, less flexible, portion of thread. The stopping mechanism acts to halt the movement of the thread as the needle and thread is pulled through the suture point, securing the remainder of the suture thread on the first side of the tissue being sutured. On this side of the stopping mechanism, a curled portion of thread is featured, such that the suture thread curls into a corkscrew configuration that loops around several times before coming to an end. This corkscrew configuration allows for a ready-made knot in the suture device, to aid in minimizing interference and obstruction during surgery. The length of the entire device, from one tip of the needle to the end of the curled suture, is of a length significantly shorter than typical needles and suture, which may stretch up to several feet long, which also aids in reducing interference and obstruction during the surgical process.

The present invention solves existing problems in the surgical field, namely providing a suture device that aids in suturing by providing a preexisting knot formation that aids the surgeon in creating a knot during the suture process, and by also providing additional features, such as varied stopping mechanisms and varied lengths of thread, to further streamline and simplify the suturing process.

### BRIEF DESCRIPTION OF DRAWINGS

Reference will now be made to the drawings, wherein like parts are designated by like numerals, and wherein:

FIG. 1A is a view of a patient with a schematic depiction of a surgical area;

FIG. 1B is a view of a patient with a schematic depiction of a surgical area, with a surgical instrument inserted in the surgical area;

FIG. 1C is a view of a laparoscopic surgery in progress, utilizing the curled suture device of the present invention;

FIG. 1D is a view of a laparoscopic surgery in progress, utilizing the curled suture device of the present invention to create a knot in the surgical area;

FIG. 2A is a schematic view of the curled suture device according to an embodiment of the present invention;

FIG. 2B is a schematic view of the curled suture device according to a second embodiment of the present invention;

FIG. 3A is a detailed view of the first step of suturing using the curled suture device of the present invention;

FIG. 3B is a detailed view of the second step of suturing using the curled suture device of the present invention; and

FIG. 3C is a detailed view of the third step of suturing using the curled suture device of the present invention.

### DETAILED DESCRIPTION OF THE DRAWINGS

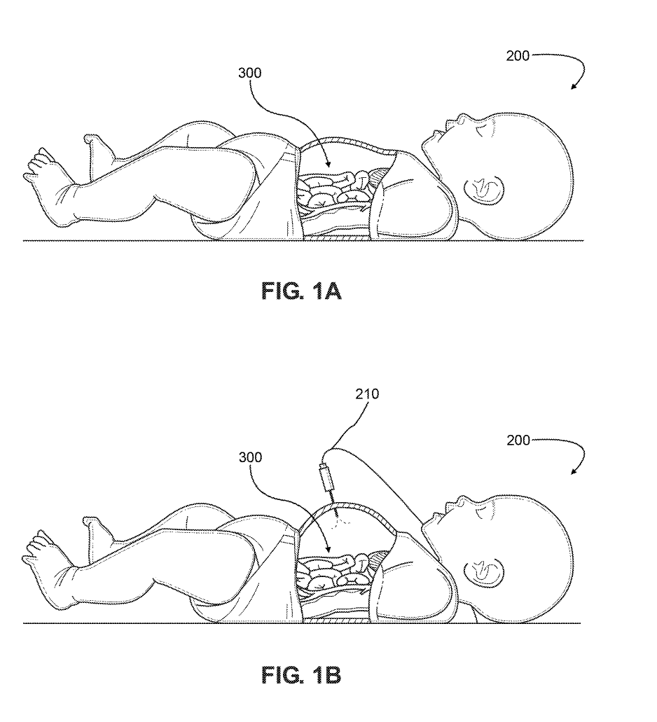

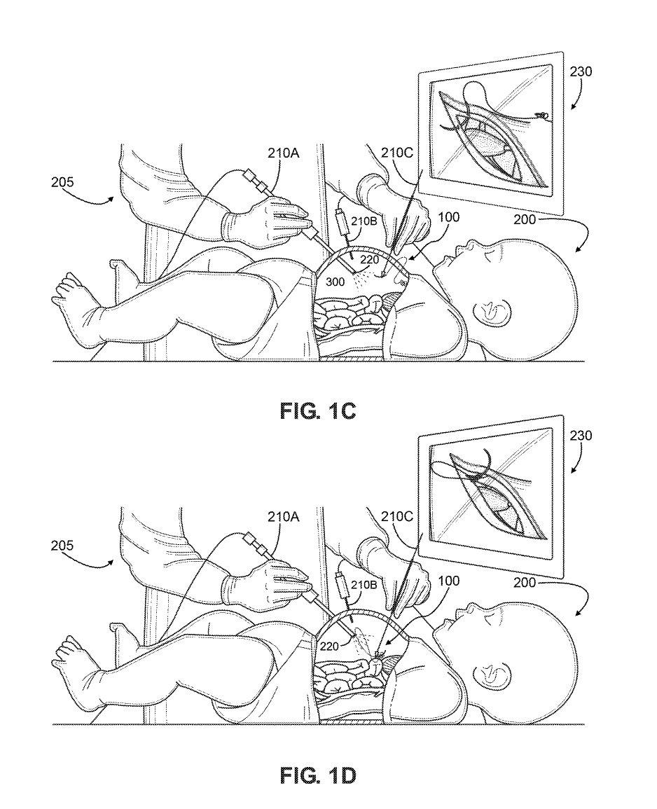

Referring to FIGS. 1A-1D, a laparoscopic surgery using the curled suture device 100 of the present invention is depicted. As shown in FIGS. 1A and 1B, the patient 200 will have a certain surgical cavity 300 wherein the surgeon will perform the procedure. In laparoscopic surgeries, the surgical cavity 300 will have various instruments 210 inserted into the surgical cavity, such as the camera, the suture, and instruments setting the surgical area in the appropriate manner. For instance, as shown in FIG. 1C, the patient 200 with surgical cavity 300 may have instruments 210A, 210B, and 210C inserted into the surgical cavity 300 to enable the surgeon 205 to perform the procedure.

As shown in FIGS. 1C and 1D, which depict a typical laparoscopic surgical procedure, there is a camera 220 focused on the appropriate area of the surgical cavity 300. This camera 220 allows the surgeon 205 to view the appropriate area of the surgical cavity 300 on a screen 230 depicting the appropriate area of the surgical cavity 300. However, the camera 220 is, by its nature, limited in scope, and will only ever be able to depict a limited view of the surgical cavity 300 on the screen 230. Accordingly, if the surgeon 205 needs to see additional areas of the surgical cavity 300, he or she will need to either continually adjust camera 220 to the appropriate area of the surgical cavity 300, which may disrupt the procedure or physicality of the patient 200, or operate outside the viewing area of the camera 220, which could lead to issues within the procedure.

However, in using the curled suture device 100 of the present invention, the camera 220 is able to present the full scope of the procedure area on the screen 230, with minimal movement of the camera 220 within the surgical cavity 300. For example, as depicted in FIG. 1D, the camera 220 may depict on the screen 230 the third step of suturing using the curled suture device 100. It may be understood by those in the art that this focusing of the camera 220 on a single area of the surgical cavity 300 is facilitated by provision of a suture device that provides for a ready-made knot, which eliminates the need for the surgeon 205 to create his or her own knot in the suture when performing the procedure.

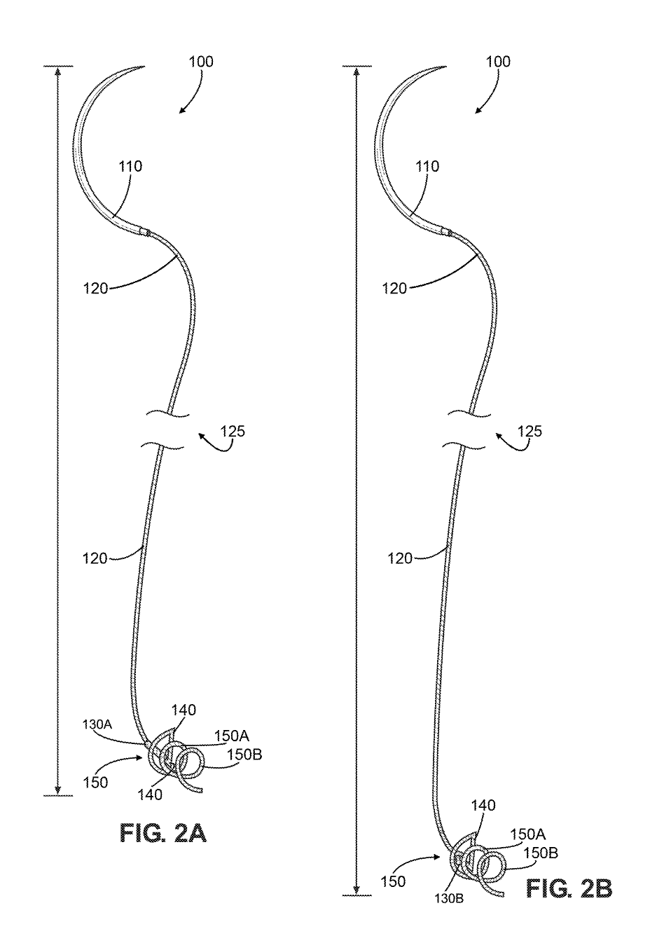

Referring now to FIG. 2, an embodiment of the curled suture device 100 of the present invention features a needle 110 at one end of the curled suture device 100, and a set of preexisting curls 150 at the opposite end of the curled suture device 100 from the needle 110. The thread 120 may be any one of several varied lengths, so that the surgeon or suturing professional may select the appropriately-sized curled suture device 100 as needed for a specific procedure. For example, it is known in the art that a surgeon may use a suture device that is 36″, or 90 cm, long for one procedure, or a shorter 6″, or 15 cm, suture device for a different procedure. However, in all instances in the prior art, the suture device must be several inches long to allow sufficient room for the surgeon to thread the appropriate tissue and appropriately knot the suture during and at the completion of the procedure to ensure that the tissue is appropriately mended. Accordingly, although the curled suture device 100 of the present invention may have a thread 120 of varied lengths, such that the gap 125 pictured in the FIGS. 2A and 2B may be of several varied lengths, the curled suture device 100 will almost always feature a thread 120 of a length shorter than a corresponding suture device without a set of curls 150, which would require additional length to prepare and create a knot in the final step of suturing the tissue.

Thread 120 may be comprised of any one of varied materials that are generally available as suturing thread materials. For example, thread 120 may be a polypropylene suture like PROLENE, which is a non-absorbable surgical suture. Other examples of a non-absorbable surgical suture are polyester sutures like ETHIBOND or nylon sutures like ETHILON or NUROLON. Such a non-absorbable suture may be preferable in certain surgical procedures when the surgeon requires a thread that will be stronger and longer-lasting than other absorbable threads, such as in cardiovascular procedures. Alternatively, thread 120 may be made of an absorbable thread, such as, without limitation, a poliglecaprone suture like MONOCRYL, a polyglactin suture like VICRYL, or a polydioxanone suture like PDS II. Such a suture may be preferable in certain procedures when it is preferable that the suture ultimately break down within the patient's body within a certain period of time. For example, certain absorbable threads may be broken down and absorbed within the patient's body in as little as two weeks, or may last over a year. However, such absorbable sutures are not preferable in procedures wherein the suture is meant to not only mend, but permanently connect, two pieces of tissue, such as repairing stitches for blood vessels or in cardiovascular or neurological procedures. The curled suture device 100 of the present invention is not meant to be limited to one type of thread 120, but may be available as either an absorbable thread or non-absorbable thread, so that the surgeon may select the appropriate type of curled suture device 100 based in part on the type of thread 120 featured in the curled suture device 100.

Additionally, thread 120 may be of either a braided or non-braided type. If thread 120 is braided, it may feature a number of strands woven together like a string. Such a braided thread, which may be made of a polyester, silk, or other material, would provide additional grip for the suture and may be stronger than a non-braided thread. However, a non-braided thread may be preferable as the thread 120 of the curled suture device 100 because it may be less prone to becoming infected, and may provide for a smoother surgical procedure within the body. Thread 120 of the curled suture device 100 of the present invention may be of either this braided or non-braided variety, so that the surgeon may select the appropriate type of curled suture device based in part on whether the thread 120 is braided or non-braided.

Needle 110 of the curled suture device 100 may also be provided in different embodiments. Suture needles are generally of two types: a cutting needle, or a tapered needle. Needle 110 of the curled suture device may be either cutting or tapered, such that the surgeon may select the appropriate type of curled suture device based in part on whether the needle 110 is a cutting or tapered needle.

Referring back to FIG. 2, the curled suture device 100 features, at the end opposite the needle 110, an end section comprising a set of pre-existing curls 150, a straight portion of thread 140 directly before the pre-existing curls 150, and a stopper 130 featured immediately prior to the straight portion of thread 140, such that the straight portion of thread 140 acts as an intermediary feature between the stopper 130 and set of pre-existing curls 150.

As shown in FIG. 2A, the curled suture device 100 may feature a stopper 130 that comprises an additional layer of material 130A directly overlaid on the suture thread, and directly adjacent the straight portion of thread 140. When the curled suture device 100 is pulled through a piece of tissue, the additional layer of material 130A will act as a stopper for the curled suture device because the area of the suture thread overlaid with the additional layer of material 130A is accordingly thicker than the main portion of thread 120. It therefore cannot be pulled through the tissue as easily as thread 120, and would require significant extra effort to be pulled through the tissue being sutured. Accordingly, when needle 110 is pulled through a piece of tissue, the main portion of thread 120, up to additional layer of material 130A, will be pulled through the piece of tissue, but the straight portion of thread 140 and set of pre-existing curls 150 will remain on the first side of the tissue due to the action of the additional layer of material 130A. It may be preferable that this additional layer of material 130A is featured on a non-absorbable suture thread, as it may otherwise interfere with an absorbable suture thread breaking down in the body appropriately, as discussed above.

Stopper knot 130B, pictured in FIG. 2B, functions in much the same manner as the additional layer of material 130A. Stopper knot 130B is featured directly adjacent the straight portion of thread 140, such that the straight portion of thread 140 acts as an intermediary feature between stopper knot 130B and set of pre-existing curls 150. Stopper knot 130B is configured such that when needle 110 and thread 120 are pulled through a piece of tissue, the stopper knot 130B will halt the progress of the thread 120 through the piece of tissue. The stopper knot 130B, straight portion of thread 140, and set of pre-existing curls 150 will therefore remain on the first side of the tissue, ready for the creation of the suture knot, discussed in more detail below. It may be preferable that this stopper knot 130B is featured on an absorbable suture thread, as it would be created with the same material as the absorbable suture thread and would thus break down in the body appropriately, according to the requirements of the absorbable suture thread selected for the procedure, as discussed above.

It may be appreciated that stopper 130 may take a variety of forms, not meant to be limited to the additional layer of material and stopper knot discussed above. For example, the stopper may be a portion of thread configured with a jagged edge, such that the jagged edge would be caught on a piece of tissue as the needle and thread are pulled through the tissue.

As shown in FIGS. 2A and 2B, the set of pre-existing curls 150 may include, without limitation, a first pre-existing curl 150A and second pre-existing curl 150B. To appropriately create a knot, as discussed below, the set of pre-existing curls 150 should at least comprise a first pre-existing curl 150A and second pre-existing curl 150B. However, it is to be understood that the set of pre-existing curls 150 may include additional pre-existing curls, such as a third or fourth curl, to allow for a larger or more secure knot, which may be preferable by the surgeon in some procedures.

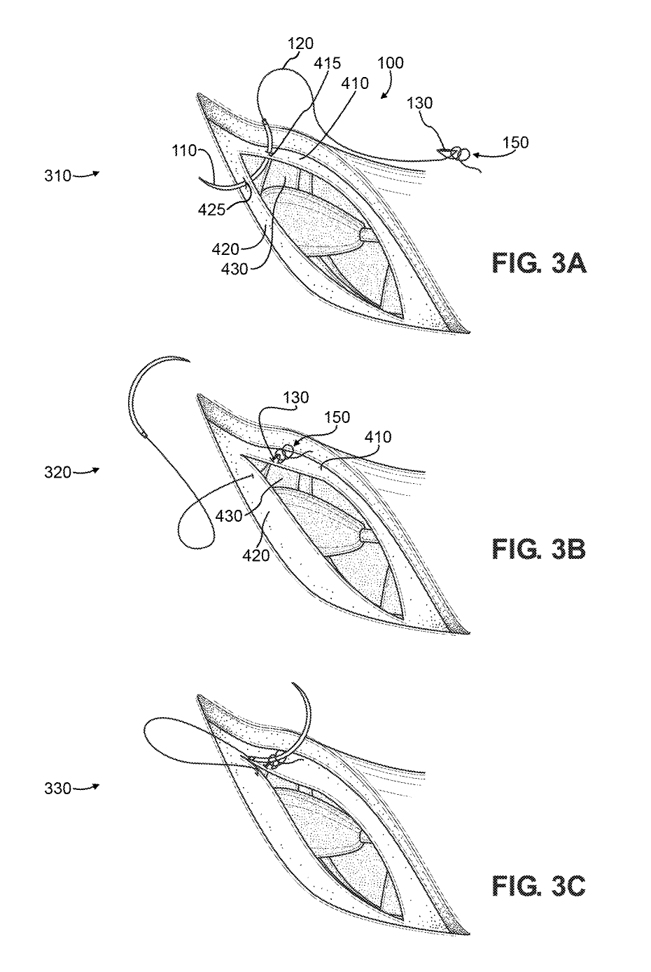

Referring now to FIGS. 3A-3C, a method of suturing using the curled suture device 100 of the present invention is depicted. In the first step 310, pictured in FIG. 3A, the needle 110 is inserted through the first piece of tissue 410 to create a first hole 415 in the first piece of tissue 410. The needle 110 is then inserted in the second piece of tissue 420 such that second hole 425 is created in the second piece of tissue 420. The needle 110 of the curled suture device 100 thus connects the first and second pieces of tissue in a traditional manner of suturing. It may be understood that although FIG. 3A depicts two pieces of tissue close enough together that the needle 110 is connected to both the first piece of tissue 410 and second piece of tissue 420 at the same time, the first and second pieces of tissue 410 and 420 may be of such a distance apart from one another that the thread 120 of the curled suture device may be connecting the first and second pieces of tissue 410 and 420 together as the needle 110 is in the process of creating the first and second holes 415 and 425 in this first step of suturing 310.

Referring now to FIG. 3B, the second step of suturing 320 consists of pulling needle 110 such a distance that the stopper 130 of the curled suture device 100 is utilized. In pulling needle 110 such a distance that stopper 130 is utilized, the gap between tissue 430 is minimized because the first piece of tissue 410 and second piece of tissue 420 are forced closer together. It may be appreciated that when the stopper 130 of the curled suture device 100 is adjacent the first hole 415 in the first piece of tissue 410, the thread 410 will no longer be pulled through the first hole 415 in the first piece of tissue 410. Instead, first piece of tissue 410 will be pulled closer to second piece of tissue 420, minimizing the gap 430 between the first and second pieces of tissue 410 and 420. Additionally, the set of curls 150 of the curled suture device 100 will not be pulled through the first hole 415 in the first piece of tissue 410, but will instead remain intact outside the first piece of tissue 410.

When the first and second pieces of tissue 410 and 420 are pulled together in the second step of suturing 320, the straight portion of the thread 140 will also remain on the outside of the first piece of tissue 410. This straight portion of the thread 140 thereby provides a space for the set of curls 150 to be more easily accessible by the surgeon in creating the knot.

The knot is created in the third step of suturing 330, depicted in FIG. 3C. In the third step of suturing 330, the surgeon leads the needle 110 from its position on the outside of the second piece of tissue 420 through the set of curls 150 featured on the outside of the first piece of tissue 410. The surgeon leads the needle 110 first through the first curl 150A, adjacent the straight portion of thread 140, and then leads the needle 110 through the second curl 150B, and any additional curls that may be featured in the set of curls 150. After the needle 110 is guided through the set of curls 150, the surgeon will pull on the needle 110, which creates a knot in the curled suture device 100. While creating the knot in the curled suture device 100, the pulling motion of the needle 110, and accordingly the thread 120, will also effectively close the gap 430 between the first and second pieces of tissue 410 and 420. Accordingly, at the end of the third step of suturing 330, the first and second pieces of tissue 410 and 420 are pulled adjacent to each other, as required by the procedure, and a knot is created holding the suture in place.

It may be appreciated that by using the curled suture device 100 of the present invention, the surgeon may effectively suture the appropriate tissue with only one hand, given that he is not required to create a knot in the suture by other means. Accordingly, if, in creating the knot in the third step of suturing 330, the surgeon realizes that the upcoming knot is in an appropriate location, the surgeon may easily adjust the location of the suture and upcoming knot within the surgical cavity. Such an adjustment is facilitated by use of the curled suture device 100 and its set of preexisting curls 150, in part because the surgeon is able control the movement of both the curled suturing device 150 and tissue 410 or 420 with only one hand.

It may also be appreciated in effectively suturing the appropriate tissue with only one hand operating the curled suture device 100, the curled suture device 100 thereby decreases the instruments and crowding that takes places in a laparoscopic surgical procedure using the suture devices presently available in the art. Moreover, the curled suture device 100 also focuses the surgical area of the procedure on the area featuring the set of curls 150, such that the surgeon may more effectively utilize his or her available viewing area, which is typically limited in a laparoscopic procedure, as discussed above and depicted in FIGS. 1C and 1D.

Many changes and modifications can be made in the present invention without departing from the spirit thereof. I therefore pray that my rights to the present invention be limited only by the scope of the appended claims.

I claim:

1. A curled suture device, comprising:
(i) a needle featured at one end of the curled suture device; and
(ii) a thread featuring a first end and a second end, wherein said first end is operatively connected to said needle, said thread further comprising:
(a) a set of curls featured at said second end of the thread, said set of curls including at least a first curl and a second curl, and wherein said set of curls have a curled configuration;
(b) a straight portion of thread having a first end and a second end, wherein said second end of said straight portion of thread is immediately adjacent said set of curls; and
(c) a stopper mechanism featured immediately adjacent said first end of said straight portion of thread;
wherein said needle is either a cutting needle or a tapered needle;
wherein said set of curls retain said curled configuration until said set of curls are transformed into a knot in said thread; and
wherein said straight portion of thread is located closer to said second end of thread than said first end of thread.

2. The curled suture device of claim 1, wherein said stopper mechanism comprises an additional layer of material directly overlaid a portion of thread immediately adjacent said first end of said straight portion of thread.

3. The curled suture device of claim 1, wherein said stopper mechanism comprises a stopper knot featured immediately adjacent said first end of said straight portion of thread.

4. The curled suture device of claim 2, wherein said thread is a non-absorbable suture thread.

5. The curled suture device of claim 3, wherein said thread is an absorbable suture thread.

6. The curled suture device of claim 2, wherein said thread is a braided thread.

7. The curled suture device of claim 2, wherein said thread is a non-braided thread.

8. The curled suture device of claim 3, wherein said thread is a braided thread.

9. The curled suture device of claim 3, wherein said thread is a non-braided thread.

10. A method of suturing, comprising:
(i) providing a curled suture device comprising:
(a) a needle featured at one end of the curled suture device; and
(b) a thread featuring a first end and a second end, wherein said first end is operatively connected to said needle, said thread further comprising:
(1) a set of curls featured at said second end of thread, said set of curls including at least a first curl and a second curl, and wherein said set of curls have a curled configuration;
(2) a straight portion of thread having a first end and a second end, wherein said second end of said straight portion of thread is immediately adjacent said set of curls; and
(3) a stopper mechanism featured immediately adjacent said first end of said straight portion of thread;
wherein said needle is either a cutting needle or a tapered needle;
wherein said set of curls retain said curled configuration until said set of curls are transformed into a knot in said thread; and
wherein said straight portion of thread is located closer to said second end of thread than said first end of thread;
(ii) inserting said needle through a first piece of tissue to create a first hole in said first piece of tissue;
(iii) pulling said needle a distance away from said first piece of tissue, such that said thread is pulled through said first hole of said first piece of tissue, and inserting said needle through a second piece of tissue to create a second hole in said second piece of tissue;
(iv) pulling said needle a distance away from said second piece of tissue, such that said thread is pulled through said second hole of said second piece of tissue until said stopper mechanism of said thread is adjacent said first hole of said first piece of tissue; and
(v) inserting said needle through said set of curls and pulling said needle a distance away from said set of curls, such that said set of curls create a knot in said thread.

11. The method of suturing of claim 10, wherein said stopper mechanism comprises an additional layer of material directly overlaid a portion of thread immediately adjacent said first end of said straight portion of thread.

12. The method of suturing of claim 10, wherein said stopper mechanism comprises a stopper knot featured immediately adjacent said first end of said straight portion of thread.

13. The method of suturing of claim 11, wherein said thread is a non-absorbable suture thread.

14. The method of suturing of claim 12, wherein said thread is an absorbable suture thread.

15. The method of suturing of claim 11, wherein said thread is a braided thread.

16. The method of suturing of claim 11, wherein said thread is a non-braided thread.

17. The method of suturing of claim 12, wherein said thread is a braided thread.

18. The method of suturing of claim 12, wherein said thread is a non-braided thread.
